SEQUENCE LISTING (7 sequences)

SEQ ID NO: 1 - DNA, 457 bp, Artificial Sequence
  Synthetic Sequence
gagctccatatgatcgaagttaaaccgatcaacgctgaagacacgtacgacctgcgtcaccgtgttctgcgtccgaaccagccgatcgaagcctgcatgttcgaatccgacctgacccgttccgctttccacctgggtggtttctacggtggtaaactgatctccgttgcttccttccaccaggctgaacactccgaactgcaaggtaaaaaacagtaccaactgcgtggtgttgctaccctggaaggttaccgtgaacagaaagctggttcctccctggttaaacacgctgaagaaatcctgcgtaaacgtggtgctgacatgatctggtgcaacgctcgtacctccgcttccggttactaccgtaaactgggtttctccgaacagggtgaagttttcgacaccccgccggttgggccccacatcctgatgtacaaacgtatcacctaattctaga

SEQ ID NO: 2 - DNA, 461 bp, Artificial Sequence
  Synthetic Sequence
gagctccggatccatgatcgaagttaaaccgatcaacgctgaagacacgtacgacctgcgtcaccgtgttctgcgtccgaaccagccgatcgaagcctgcatgttcgaatccgacctgacccgttccgctttccacctgggtggtttctacggtggtaaactgatctccgttgcttccttccaccaggctgaacactccgaactgcaaggtaaaaaacagtaccaactgcgtggtgttgctaccctggaaggttaccgtgaacagaaagctggttcctccctggttaaacacgctgaagaaatcctgcgtaaacgtggtgctgacatgatctggtgcaacgctcgtacctccgcttccggttactaccgtaaactgggtttctccgaacagggtgaagttttcgacaccccgccggttgggccccacatcctgatgtacaaacgtatcacctaattctaga

SEQ ID NO: 3 - protein, 146 aa, Artificial Sequence
  Synthetic Sequence
MIEVKPINAEDTYDLRHRVLRPNQPIEACMFESDLTRSAFHLGGFYGGKLISVASFHQAEHSELQGKKQYQLRGVATLEGYREQKAGSSLVKHAEEILRKRGADMIWCNARTSASGYYRKLGFSEQGEVFDTPPVGPHILMYKRIT

SEQ ID NO: 4 - DNA, 143 bp, Artificial Sequence
  Synthetic Sequence
gagctcgctcccccgccgtcgttcaatgagaatggataagaggctcgtgggattgacgtgagggggcagggatggctatatttctgggagttacgtttccacctcaaagtgaaatatagtatagttgtagggagggacatatg

SEQ ID NO: 5 - DNA, 150 bp, Artificial Sequence
  Synthetic Sequence
gagctcgctcccccgccgtcgttcaatgagaatggataagaggctcgtgggattgacgtgagggggcagggatggctatatttctgggagtcgagtagaccttgttgttgtgaaaattcttaattcatgagttgtagggagggacatatg

SEQ ID NO: 6 - DNA, 140 bp, Artificial Sequence
  Synthetic Sequence
gagctcgctcccccgccgtcgttcaatgagaatggataagaggctcgtgggattgacgtgagggggcagggatggctatatttctgggagttacgtttccacctcaaagtgaaatatagtatagttgtagggagggatcc

SEQ ID NO: 7 - DNA, 226 bp, Artificial Sequence
  Synthetic Sequence
tctagagtagacattagcagataaattagcaggaaataaagaaggataacgagaaagaactcaagtaattatccttcgttctcttaattgaattgcaattaaactcggcccaatcttttactaaaaggattgagccgaatacaacaaagattctattgcatatattttgactaagtatatacttacctagatatacaagatttgaaatacaaaatctagcaagctt

(19) United States
(12) Patent Application Publication
(10) Pub. No.: US 2008/0044904 A1
(43) Pub. Date: Feb. 21, 2008

(54) Glyphosate N-acetyltransferase (GAT) confers glyphosate resistance when expressed in plastids

(76) Inventor: Pal Maliga, East Brunswick, NJ (US)

Correspondence Address: DANN, DORFMAN, HERRELL & SKILLMAN, 1601 MARKET STREET, SUITE 2400, PHILADELPHIA, PA, 19103-2307, US

(21) Appl. No.: 11/509,457

(22) Filed: Aug. 24, 2006

Related U.S. Application Data
(60) Provisional application No. 60/710,970, filed on Aug. 24, 2005.

(51) Int. Cl. A01H 5/00; C07H 21/04; C12N 15/00; C12N 5/00

(52) U.S. Cl. 435/419; 435/320.1; 536/23.2; 536/24.1; 800/298

(57) ABSTRACT

Constructs for the expression of glyphosate N-acetyltransferase in plastids and conferring glyphosate resistance are disclosed.

This application claims priority to U.S. Provisional Application 60/710,970, filed Aug. 24, 2005. The entire disclosure of this application is incorporated by reference herein.

### FIELD OF THE INVENTION

This invention relates to the fields of glyphosate resistance and transgenic plants.

### BACKGROUND OF THE INVENTION

Several patent documents and research articles are cited throughout this application in order to more fully describe the state of the art to which this invention pertains. The disclosure of each of these citations is incorporated by reference herein.

Glyphosate is an environmentally friendly herbicide which is widely used for weed control of transgenic crops expressing a 5-enolpyruvylshikimate-3-phosphate synthase (EPSP synthase or EPSPS) transgene in the nucleus which is insensitive to inhibition by glyphosate. In order to provide protection from glyphosate, the EPSPS gene should be expressed at high levels in plastids (Ye et al. (2001) Plant J., 25:261-270). Recently, a novel glyphosate resistance gene was described encoding a glyphosate modifying enzyme, glyphosate N-acetyltransferase (GAT) (Castle et al. (2004) Science, 304:1151-1154). The native forms of this enzyme identified in Bacillus licheniformis are not suitable to confer glyphosathe tolerance to plants. However, DNA shuffling of the enzyme significantly improved GAT kinetic properties so that it confers glyphosate tolerance to crops. Use of GAT to protect crops from glyphosate is described in pending US Patent Application Publication Nos: 20050246798, 20050060767, 20040082770, 20030148309, 20030083480, and 20020058249. Notably, these patent applications fail to demonstrate glyphosate resistance by expressing GAT in plastids.

### SUMMARY OF THE INVENTION

It is an object of the invention to provide tools and protocols for the expression of GAT in all plastid types, including but not limited to proplastids, chloroplasts, amyloplasts and elaioplasts that will confer resistance to the herbicide glyphosate (e.g., glyphosate-isopropylammonium).

In accordance with one aspect, isolated nucleic acid molecules encoding a glyphosate N-acetyltransferase for expression in a plant plastid are provided. In a particular embodiment, the glyphosate N-acetyltransferase has at least 75%, 80%, 85%, 90%, 95%, 97%, or 100% homology to SEQ ID NO: 3. The nucleic acid molecule encoding a glyphosate N-acetyltransferase may have at least 75%, 80%, 85%, 90%, 95%, 97%, or 100% homology to SEQ ID NO: 1 or SEQ ID NO: 2.

In another embodiment of the invention, vectors comprising a nucleic acid molecule encoding a glyphosate N-acetyltransferase operably linked to a promoter suitable for expression in plastids are provided. The promoter may have at least 75%, 80%, 85%, 90%, 95%, 97%, or 100% homology to a sequence selected from the group consisting of SEQ ID NO: 4, SEQ ID NO: 5, and SEQ ID NO: 6. The vectors may also comprise a terminator. An exemplary terminator is the rbcL gene 3′-untranslated region. In a particular embodiment, the rbcL gene 3′-untranslated region has at least 75%, 80%, 85%, 90%, 95%, 97%, or 100% homology with SEQ ID NO: 7. In a particular embodiment, plant cells and plants comprising the vectors of the instant invention are also provided. The vectors of the invention may also include a multiple cloning site for the insertion of at least one nucleic acid encoding a heterologous protein of interest.

In accordance with another aspect, the instant invention provides promoter sequences for the expression of a protein conferring glyphosate resistance in a higher plant comprising a sequence having at least 75%, 80%, 85%, 90%, 95%, 97%, or 100% homology with a sequence selected from the group consisting of SEQ ID NO: 4, SEQ ID NO: 5, and SEQ ID NO: 6.

### BRIEF DESCRIPTION OF THE DRAWINGS

FIG. 1 provides the protein sequence of GAT.

FIGS. 2A and 2B provide the GATpt1n coding region contained in an NdeI-XbaI fragment and the GATpt1b coding region contained in a BamHI-XbaI fragment.

FIG. 3 provides the sequences of the PrrnPclpPrbcL2, PrrnLrbcL2n, and PrrnPclpPrbcL1 promoters.

FIG. 4 provides the sequence of TrbcL.

FIG. 5 shows a map of plastid transformation vectors and the transformed region of the plastid genome (ptDNA). (A) Map of the plastid targeting region of plastid vectors pMHB125 and pMHB127. Shown are the left and right targeting regions (LTR, RTR), the GAT gene and selective marker aadA/gfp, and plastid genes rps12/7, trnV and rrn16. Below is the cognate region of wild type ptDNA; crossed lines symbolize integration by two homologous recombination events. Above the line are listed relevant restriction sites; sites removed during constriction are in brackets. Note that restriction sites in vectors pMHB125 and pMHB127 are identical. Below the wild type map are shown the position of the 2.0 kb EcoRV-ApaI probe and 3.3 kb wild-type BamHI fragment. (B) Map of ptDNA transformed with plasmid pMHB126. (C) Map of ptDNA transformed with pMHB125 or pMHB127. Position of hybridizing 6.1 kb band is shown below. DNA sequence of wild-type ptDNA is found under GenBank Accession No. Z00044. The plastid vectors is a pPRV111 vector derivative (U.S. Pat. No. 5,877,402), which carries the aadA-gfp gene from plasmid pMSK56 (US application No. 20060150287) replacing the aadA gene.

FIG. 6 depicts DNA gel blot analysis which confirms uniform transformation of ptDNA with GAT vectors. Total cellular DNA was digested with the BamHI restriction enzyme and probed with the 2.0 kb EcoRV-ApaI fragment shown in FIG. 1A. Note absence of 3.3 kb wild type fragment in plants transformed with plasmids Nt-pMHB125 and Nt-pMHB127, and presence of the 6.1 kb fragment containing the GAT gene. DNA of pMSK56-transformed plants is shown alongside as a reference, in which the probe detects a smaller 5.2 kb fragment lacking the GAT gene.

FIG. 7 demonstrates that tobacco cells expressing GAT in chloroplasts are resistant to 100 μM glyphosate in the RMOP culture medium. Shown are three-week-old cultures initiated from Nt-pMHB125 and Nt-pMHB126 leaves and wild type control. The culture medium and general tissue culture protocols are described in references Svab and Maliga, 1993; and Lutz et al., 2006.

### DETAILED DESCRIPTION OF THE INVENTION

The following definitions are provided to aid in understanding the subject matter regarded as the invention.

The term “glyphosate,” as used herein, includes any herbicidally effective form of N-phosphonomethylglycine (such as any ester or salt thereof, including, for example, glyphosate-diammonium, glyphosate-isopropylammonium, glyphosate-monoammonium, glyphosate-potassium, glyphosate-sesquisodium, glyphosate-trimesium) and other forms which result in the production of the glyphosate anion in plants.

“Heteroplastomic” refers to the presence of a mixed population of different plastid genomes within a single plastid or in a population of plastids contained in plant cells or tissues.

“Homoplastomic” refers to a pure population of plastid genomes, either within a plastid or within a population contained in plant cells and tissues. Homoplastomic plastids, cells or tissues are genetically stable because they contain only one type of plastid genome. Hence, they remain homoplastomic even after the selection pressure has been removed, and selfed progeny are also homoplastomic. For purposes of the present invention, heteroplastomic populations of genomes that are functionally homoplastomic (i.e., contain only minor populations of wild-type DNA or transformed genomes with sequence variations) may be referred to herein as “functionally homoplastomic” or “substantially homoplastomic.” These types of cells or tissues can be readily purified to a homoplastomic state by continued selection.

“Plastome” refers to the genome of a plastid.

“Transplastome” refers to a transformed plastid genome.

Transformation of plastids refers to the stable integration of transforming DNA into the plastid genome that is transmitted to the seed progeny of plants containing the transformed plastids.

“Selectable marker gene” refers to a nucleic acid sequence that upon expression confers a phenotype by which successfully transformed plastids or cells or tissues carrying the transformed plastid can be identified.

“Transforming DNA” refers to homologous DNA, or heterologous DNA flanked by homologous DNA, which when introduced into plastids becomes part of the plastid genome by homologous recombination.

An alternative type of transforming DNA refers to a DNA which contains recombination site sequences for a site-specific recombinase or integrase. Insertion of this type of DNA is not dependent on the degree of homology between the transforming DNA and the plastid to be transformed but rather is catalyzed by the action of the recombinase or integrase on the first and second recombination sites.

“Operably linked” refers to two different regions or two separate nucleic acid sequences spliced together in a construct such that both regions will function to promote gene expression and/or protein translation.

“Nucleic acid” or a “nucleic acid molecule” as used herein refers to any DNA or RNA molecule, either single or double stranded and, if single stranded, the molecule of its complementary sequence in either linear or circular form. In discussing nucleic acid molecules, a sequence or structure of a particular nucleic acid molecule may be described herein according to the normal convention of providing the sequence in the 5′ to 3′ direction. With reference to nucleic acids of the invention, the term “isolated nucleic acid” is sometimes used. This term, when applied to DNA, refers to a DNA molecule that is separated from sequences with which it is immediately contiguous in the naturally occurring genome of the organism in which it originated. For example, an “isolated nucleic acid” may comprise a DNA molecule inserted into a vector, such as a plasmid or virus vector, or integrated into the genomic DNA of a prokaryotic or eukaryotic cell or host organism.

When applied to RNA, the term “isolated nucleic acid” refers primarily to an RNA molecule encoded by an isolated DNA molecule as defined above. Alternatively, the term may refer to an RNA molecule that has been sufficiently separated from other nucleic acids with which it would be associated in its natural state (i.e., in cells or tissues). An isolated nucleic acid (either DNA or RNA) may further represent a molecule produced directly by biological or synthetic means and separated from other components present during its production.

The phrase “heterologous molecule” refers to a molecule which is produced in the plant following introduction of a nucleic acid of the invention. Such molecules include RNA, (e.g., siRNA) and proteins.

The terms “percent similarity”, “percent identity” and “percent homology” when referring to a particular sequence are used as set forth in the University of Wisconsin GCG software program.

The term “functional” as used herein implies that the nucleic or amino acid sequence is functional for the recited assay or purpose.

The phrase “consisting essentially of” when referring to a particular nucleotide or amino acid means a sequence having the properties of a given SEQ ID No:. For example, when used in reference to an amino acid sequence, the phrase includes the sequence per se and molecular modifications that would not affect the basic and novel characteristics of the sequence.

A “vector” is a replicon, such as a plasmid, cosmid, bacmid, phage or virus, to which another genetic sequence or element (either DNA or RNA) may be attached so as to bring about the replication of the attached sequence or element.

An “expression operon” refers to a nucleic acid segment that may possess transcriptional and translational control sequences, such as promoters, enhancers, translational start signals (e.g., ATG or AUG codons), polyadenylation signals, terminators, and the like, and which facilitate the production of a polypeptide coding sequence in a host cell or organism. Such expression signals may be combined such that production of said polypeptide occurs transiently or is produced stably over the life of the cell.

The term “oligonucleotide,” as used herein refers to primers and probes of the present invention, and is defined as a nucleic acid molecule comprised of two or more ribo or deoxyribonucleotides, preferably more than three. The exact size of the oligonucleotide will depend on various factors and on the particular application and use of the oligonucleotide.

The term “probe” as used herein refers to an oligonucleotide, polynucleotide or nucleic acid, either RNA or DNA, whether occurring naturally as in a purified restriction enzyme digest or produced synthetically, which is capable of annealing with or specifically hybridizing to a nucleic acid with sequences complementary to the probe. A probe may be either single stranded or double stranded. The exact length of the probe will depend upon many factors, including temperature, source of probe and use of the method. For example, for diagnostic applications, depending on the complexity of the target sequence, the oligonucleotide probe typically contains 15 25 or more nucleotides, although it may contain fewer nucleotides. The probes herein are selected to be “substantially” complementary to different strands of a particular target nucleic acid sequence. This means that the probes must be sufficiently complementary so as to be able to “specifically hybridize” or anneal with their respective target strands under a set of pre-determined conditions. Therefore, the probe sequence need not reflect the exact complementary sequence of the target. For example, a non complementary nucleotide fragment may be attached to the 5′ or 3′ end of the probe, with the remainder of the probe sequence being complementary to the target strand. Alternatively, non complementary bases or longer sequences can be interspersed into the probe, provided that the probe sequence has sufficient complementarity with the sequence of the target nucleic acid to anneal therewith specifically.

The term “primer” as used herein refers to an oligonucleotide, either RNA or DNA, either single stranded or double stranded, either derived from a biological system, generated by restriction enzyme digestion, or produced synthetically which, when placed in the proper environment, is able to functionally act as an initiator of template-dependent nucleic acid synthesis. When presented with an appropriate nucleic acid template, suitable nucleoside triphosphate precursors of nucleic acids, a polymerase enzyme, suitable cofactors and conditions such as a suitable temperature and pH, the primer may be extended at its 3′ terminus by the addition of nucleotides by the action of a polymerase or similar activity to yield an primer extension product. The primer may vary in length depending on the particular conditions and requirement of the application. For example, in diagnostic applications, the oligonucleotide primer is typically 15 25 or more nucleotides in length. The primer must be of sufficient complementarity to the desired template to prime the synthesis of the desired extension product, that is, to be able anneal with the desired template strand in a manner sufficient to provide the 3′ hydroxyl moiety of the primer in appropriate juxtaposition for use in the initiation of synthesis by a polymerase or similar enzyme. It is not required that the primer sequence represent an exact complement of the desired template. For example, a non complementary nucleotide sequence may be attached to the 5′ end of an otherwise complementary primer. Alternatively, non complementary bases may be interspersed within the oligonucleotide primer sequence, provided that the primer sequence has sufficient complementarity with the sequence of the desired template strand to functionally provide a template primer complex for the synthesis of the extension product.

The terms “transform”, “transfect”, “transduce”, shall refer to any method or means by which a nucleic acid is introduced into a cell or host organism and may be used interchangeably to convey the same meaning. Such methods include, but are not limited to, transfection, electroporation, microinjection, PEG-fusion, biolistic bombardment and the like.

A “clone” or “clonal cell population” is a population of cells derived from a single cell or common ancestor by mitosis.

A “cell line” is a clone of a primary cell or cell population that is capable of stable growth in vitro for many generations.

The following Example is provided to describe certain embodiments of the invention. It is not intended to limit the invention in any way.

### EXAMPLE I

Plastid GAT Genes

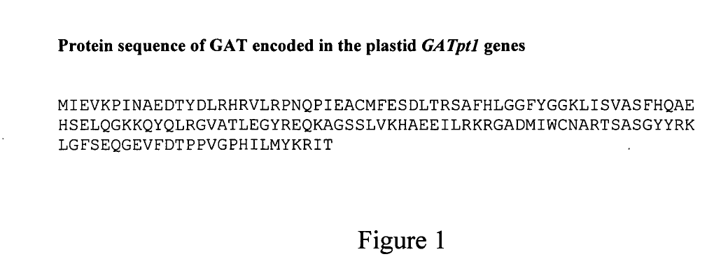

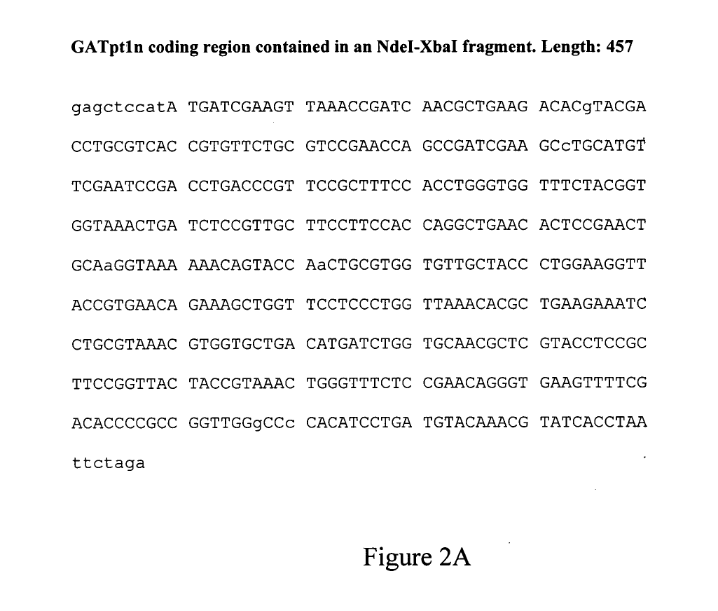

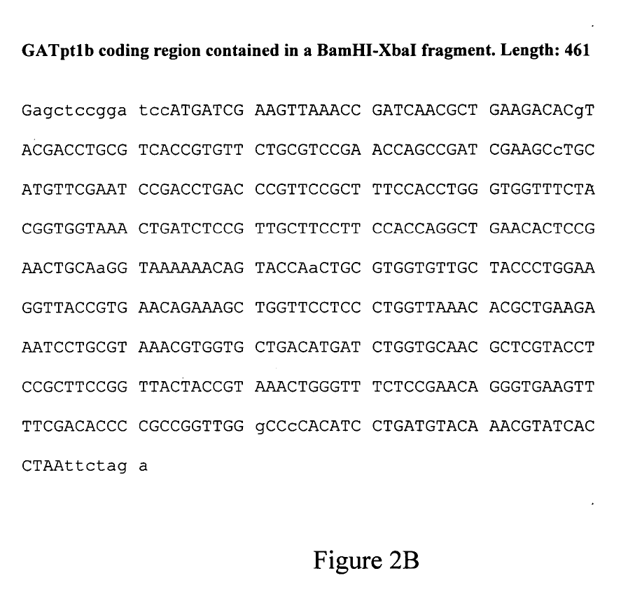

GAT genes have been designed for expression in plastids based on the protein sequence published in Castle et al. (Castle et al. (2004) Science, 304:1151-1154; GenBank Accession No. AY597418; SEQ ID NO: 3; FIG. 1). The plastid GAT GAT1pt1n coding region is contained in an NdeI-XbaI fragment (SEQ ID NO: 1; FIG. 2A). In a variant, the GATpt1b coding region is contained in a BamHI-XbaI fragment (SEQ ID NO: 2; FIG. 2B). Both plastid GAT genes encode the same protein shown in SEQ ID NO: 3 (FIG. 1) and confer resistance to glyphosate.

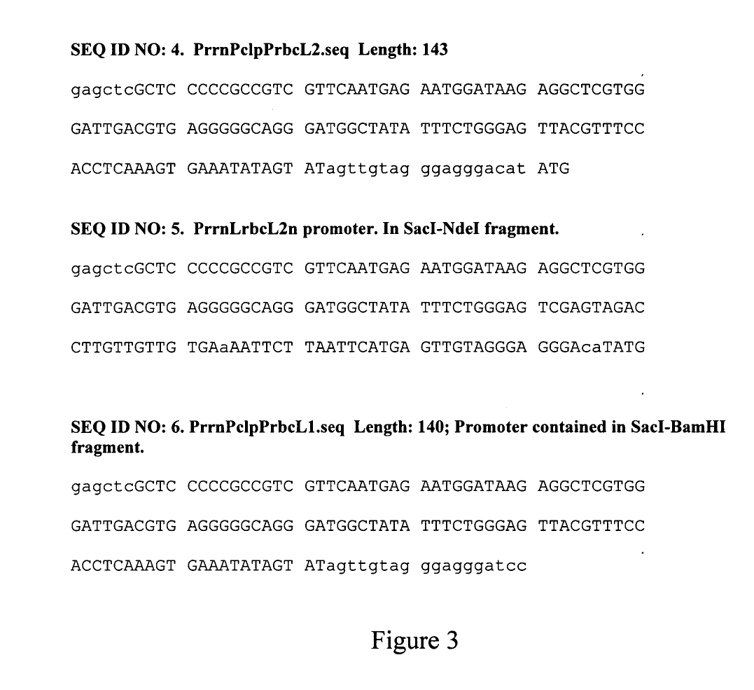

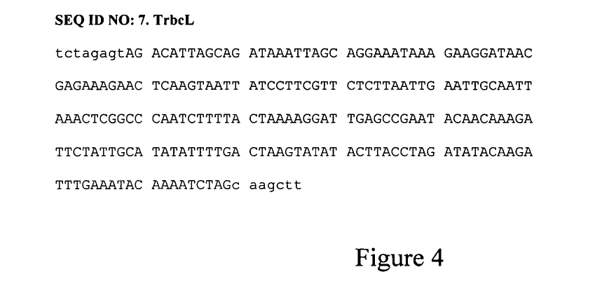

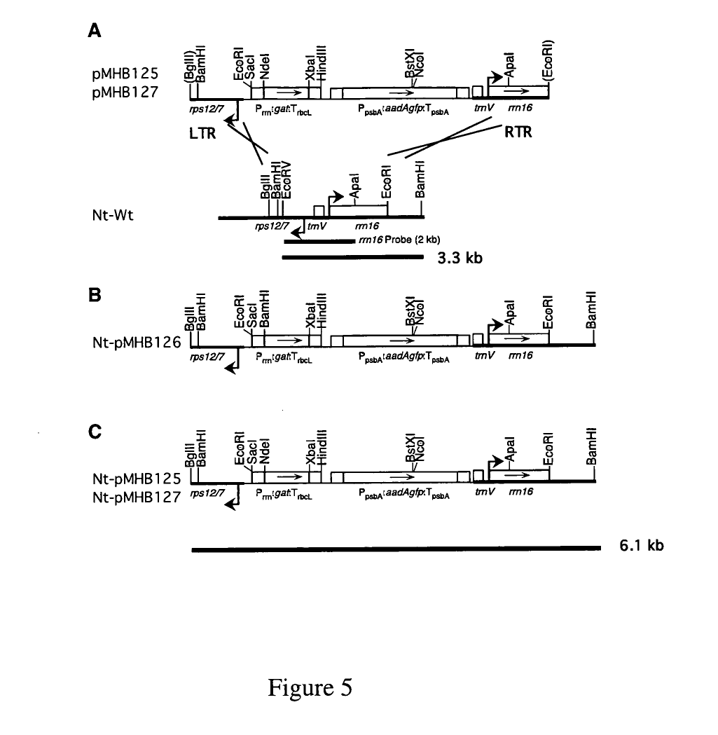

Suitable promoters for the expression of GAT are contained in SacI-NdeI (SEQ ID NO: 4 and SEQ ID NO: 5; FIG. 3) or SacI-BamHI fragments (SEQ ID NO: 6; FIG. 3). The PrrnPclpP1Lrbc12 dual promoter consists of the plastid rRNA operon PEP promoter (Suzuki et al. (2003) Plant Cell, 15:195-205; U.S. Patent Application Publication No. 20040221338) fused with the clpP1-53 NEP promoter (Sriraman et al. (1998) Nucleic Acids Res., 26:4874-4879; U.S. Patent Application Publication No. 20040040058) and the rbcL gene ribosome binding site (see SEQ ID NO: 4). The PrrnLrbcL2n promoter was obtained by fusing the plastid rRNA operon promoter with the plastid rbcL gene leader, as in plasmid pHK34 (Kuroda and Maliga (2001) Plant Physiol., 125:430-436), and modifying sequences upstream of the translation initiation codon ATG to encode an NdeI site, as in plasmid pTT40 (promoter sequence is shown as SEQ ID NO: 5). The PrrnPclpPrbcL1 promoter is similar to the PrrnPclpP1Lrbc12 promoter except that it has at its 3′-end a BamHI cloning site (SEQ ID NO: 6). Promoters contained in a SacI-NdeI fragment (SEQ ID NOs: 4 and 5) can be operably linked with the GATpt1n coding segment via the NdeI cloning site (see SEQ ID NO: 1). The promoter contained in the SacI-BamHI fragment (SEQ ID NO: 6) can be combined with GATpt1b (SEQ ID NO: 2) via the BamHI site. To provide stability for the GAT mRNAs, the rbcL gene 3′-untranslated region (TrbcL) is cloned downstream as and XbaI-HindIII fragment (SEQ ID NO: 7; U.S. Pat. No. 5,877,402; FIG. 4). Plastid GAT gene can be readily assembled using the plastid promoter—GAT coding region and TrbcL modular units, cloned into a plastid transformation vector, and introduced into plastids. Plastid transformation vectors pMHB125, pMHB126 and pMHB127 with specific GAT genes are listed in Table 1. The maps of GAT transformation vectors carrying the aadA-gfp marker genes are shown in FIG. 5.

TABLE 1
GAT genes and plasmids.

| | | | GAT |
|---|---|---|---|
| Plastid vector | GAT promoter | GAT coding | Terminator |
| pMHB125 | PrrnPclpP1Lrbcl2 | GAT1pt1n | TrbcL |
| | Seq. ID No. 4 | SEQ ID. No 1 | SEQ. ID. No. 7 |
| pMHB126 | PrrnPclpPrbcL1 | GATpt1b | TrbcL |
| | Seq. ID No. 6 | SEQ. ID. No. 2 | SEQ. ID. No. 7 |
| pMHB127 | PrrnLrbcL2n | GAT1pt1n | TrbcL |
| | Seq. ID No. 5 | (SEQ ID. No 1 | SEQ. ID. No. 7 |

Particularly useful alternative vector for the introduction of GAT genes would be the pPRV111 series, which also carry a spectinomycin resistance (aadA) marker gene, described by Zoubenko et al (Zoubenko et al. (1994) Nucleic Acids Res., 22:3819-3824; U.S. Pat. No. 5,877,402). Marker-free transplastomic plants can also be obtained using plastid transformation vectors that enable post-integration excision of marker genes from the plastid genome (Lutz et al. (2006) Nature Protocols 1(2):900-910). Also see U.S. patent application Ser. No. 10/088,634 to Maliga et al.

Transplastomic Plants with GAT Genes

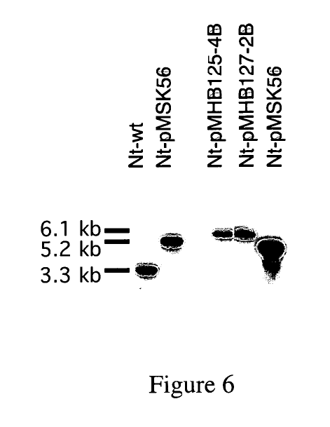

To obtain transplastomic tobacco, the transforming DNA was introduced into chloroplasts by the biolistic process where the transgenes incorporated into the plastid genome by two homologous recombination events (Svab and Maliga (1993) Proc. Natl. Acad. Sci. USA, 90:913-917; Lutz et al. (2006) Nature Protocols 1(2):900-910). An alternative method for the introduction of transforming DNA would be PEG-mediated DNA delivery (Koop and Kofer (1995) Plastid transformation by polyethylene glycol treatment of protoplasts and regeneration of transplastomic tobacco plants. In Gene transfer to plants. (Potrykus, I. and Spangenberg, G., eds). Berlin-Heidelberg-New York: Sprienger Verlag, pp. 75-82). The transplastomic clones were selected by spectinomycin resistance, plants were regenerated twice from leaf sections to obtain uniform transformation of all plastid genome copies, and subjected to DNA blot analyses. Homoplastomic clones were obtained by transformation with each, the pMHB125, pMHB126 and pMHB127 plasmids. Southern analyses of BamHI digested total cellular DNA was carried out using the targeting sequence as the probe. The results in FIG. 6 confirm the presence of the transplastomic 6.07 kb fragment and the absence of 3.3 kb wild-type fragment in Nt-pMHB125 and Nt-pMHB127 leaves. Detailed protocols for the construction and characterization of transplastomic plants are provided in Lutz et al. (Nature Protocols (2006) 1(2) :900-910).

GAT Genes Confer Glyphosate Resistance to Plants

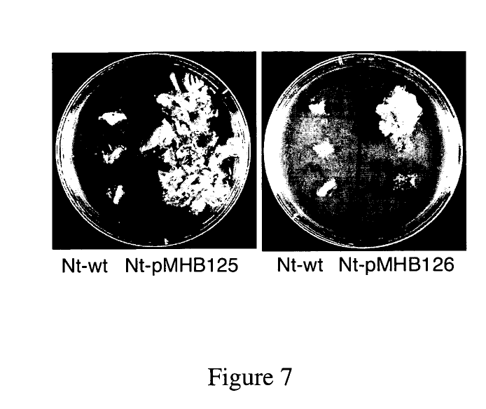

The ability of leaf sections or stem sections of plants to germinate and/or grow as green plants in a culture containing selective levels of herbicides is a reliable assay of glyphosate resistance (Ye et al. (2003) Plant Physiol., 133:402-410; Ye et al. (2001) Plant J., 25:261-270). Selective levels of glyphosate in the culture medium were in the range of 50 μm to 200 μm, to which plants expressing the CP4 EPSPS gene were resistant. Therefore, sections of the Nt-pMHB125, Nt-pMHB126 and Nt-pMHB127 leaves were inoculated onto RMOP medium containing 100 μm glyphosate (Glyphosate-isopropylammonium). The dramatic results of such a leaf test are shown in FIG. 7. Sections of Nt-pMHB125 and Nt-pMHB126 leaves formed abundantly proliferating green calli in the presence of the herbicide while the wild-type leaf sections failed to grow and bleached. Such high level of resistance was sufficient to protect the plants from glyphosate treatment under filed conditions. The new glyphosate resistance (GAT) genes, when expressed in plastids, enable efficient weed control with an environmentally friendly herbicide. Since plastids are not transmitted via pollen in most crops, the GAT gene will not be disseminated via pollen, providing an efficient containment tool (Bock, R. (2001) J. Mol. Biol., 312:425-438; Maliga, P. (2004) Annu. Rev. Plant Biol., 55:289-313).

While certain of the preferred embodiments of the present invention have been described and specifically exemplified above, it is not intended that the invention be limited to such embodiments. Various modifications may be made thereto without departing from the scope and spirit of the present invention, as set forth in the following claims.

What is claimed is:

1. An isolated nucleic acid encoding a glyphosate N-acetyltransferase for expression in a plant plastid.

2. The nucleic acid sequence of claim 1, wherein said glyphosate N-acetyltransferase has at least 90% homology to SEQ ID NO: 3.

3. The nucleic acid sequence of claim 2, wherein said glyphosate N-acetyltransferase is SEQ ID NO: 3.

4. The nucleic acid of claim 1, wherein said nucleic acid has at least 90% homology to SEQ ID NO: 1 or SEQ ID NO: 2.

5. The nucleic acid of claim 4, wherein said nucleic acid is SEQ ID NO: 1.

6. The nucleic acid of claim 4, wherein said nucleic acid is SEQ ID NO: 2.

7. A vector comprising the nucleic acid of claim 1 operably linked to a promoter.

8. The vector of claim 7 further comprising at least one nucleic acid encoding a heterologous protein of interest.

9. The vector of claim 7, which comprises a promoter having at least 90% homology with a sequence selected from the group consisting of SEQ ID NO: 4, SEQ ID NO: 5, and SEQ ID NO: 6.

10. The vector of claim 9, which comprises a promoter selected from the group consisting of SEQ ID NO: 4, SEQ ID NO: 5, and SEQ ID NO: 6.

11. The vector of claim 7, which comprises the rbcL gene 3′-untranslated region.

12. The vector of claim 7, wherein rbcL gene 3′-untranslated region has at least 90% homology with SEQ ID NO: 7.

13. A promoter sequence for expression of a protein conferring glyphosate resistance in a higher plant comprising a sequence having at least 90% homology with a sequence selected from the group consisting of SEQ ID NO: 4, SEQ ID NO: 5, and SEQ ID NO: 6.

14. The promoter sequence of claim 13, wherein said promoter is selected from the group consisting of SEQ ID NO: 4, SEQ ID NO: 5, and SEQ ID NO: 6.

15. A plant cell comprising the vector of claim 7.

16. A plant comprising the plant cell of claim 15.
